(19) United States
(12) Patent Application Publication
(10) Pub. No.: US 2024/0150729 A1
(43) Pub. Date: May 9, 2024

(54) A CLOSED-SYSTEM UPSTREAM MANUFACTURING PROCESS FOR DENGUE VIRUS PRODUCTION

(71) Applicant: Merck Sharp & Dohme LLC, Rahway, NJ (US)

(72) Inventors: Pooja Chadha, Lansdale, PA (US); Kent Harley Hamaker, Round Rock, TX (US); Shannon Haughney, Harleysville, PA (US); Samantha J. Marrone, Lansdale, PA (US); Christopher Ton; Igor Zabrodin, North Wales, PA (US)

(73) Assignee: Merck Sharp & Dohme LLC, Rahway, NJ (US)

(21) Appl. No.: 18/550,348

(22) PCT Filed: Mar. 17, 2022
(86) PCT No.: PCT/US2022/020676

§ 371 Date: Sep. 13, 2023

Related U.S. Application Data
(60) Provisional application No. 63/163,079, filed on Mar. 19, 2021.

(51) Int. Cl. C12N 7/00

(52) U.S. Cl. CPC C12N 7/00; C12N 2770/24121; C12N 2770/24152; C12N 2770/24162

(57) ABSTRACT

The instant invention discloses a closed-system, serum-free, microcarrier-based up-stream process to produce dengue virus. The process comprises a closed-system cell expansion comprising cell passages taking place in closed-system containers. These cell expansions represent a closed process, with replacement media, a cell-detachment agent, and quench medium added though sterile, weldable tubing (a closed-system environment) to eliminate all open aseptic processing following the initial vial thaw of the adherent cells. The viral production takes place in a closed-system bioreactor in adherent cell culture grown on microcarriers providing sufficient cell mass to support the production of dengue virus.

### FIELD OF THE INVENTION

The present invention relates to a scalable, serum-free, microcarrier-based closed-system process for the upstream manufacture of dengue virus.

### BACKGROUND OF THE INVENTION

The manufacture of virus and virus vaccines is well known in the art. Viral vaccines have been manufactured for human use since the 18th century with continuous improvements to vaccine manufacturing made during the last 300 years (Plotkin, S. Proc. Natl. Acad. Sci. U.S.A. Aug. 26, 2014 111 (34) 12283-1228). During this time, the state of the manufacturing art has moved from the use of primary cell cultures to continuous cell lines, the most common and most accepted of which is the Vero cell line (P Noel Barrett, Wolfgang Mundt, Otfried Kistner & M Keith Howard (2009) Expert Review of Vaccines, 8:5, 607-618). To further optimize vaccine production with the Vero cell line, closed-systems for the manufacture of virus vaccines have been disclosed (Johannes C. M. van der Loo, J. Fraser Wright, Progress and challenges in viral vector manufacturing, Human Molecular Genetics, Volume 25, Issue R1, 15 Apr. 2016, Pages R42-R52). A closed-system for the manufacture of virus vaccines reduces the risk of contamination from any number of sources. To date, there have been disclosures that highlight the benefits of a closed-system for viral manufacturing, however, most do not extend this closed-system strategy to upstream manufacturing, including cell expansion efforts (Sheu, Jonathan et al. “Large-scale production of lentiviral vector in a closed-system hollow fiber bioreactor.” Molecular therapy. Methods & Clinical Development vol. 2 15020. 17 Jun. 2015, doi:10.1038/mtm.2015.20).

In addition to the implementation of closed-system processing in the field, there have also been recent advancements in the art toward process scale up, focused primarily on bioreactor-based processes. When dealing with an adherent cell line for vaccine production in bioreactors, a surface is required for cell growth which has classically relied on microcarrier-based technologies. Microcarriers provide a solid substrate for adherent cell growth that can be suspended in cell culture medium in a bioreactor. This provides a useful alternative to traditional static cell culture surfaces such as T-flasks. Microcarrier-based vaccine production systems have been previously demonstrated in the art. These systems allow for scale up in culture size based on the volume of bioreactor chosen rather than the need to scale-out in number of flasks, for static cell culture growth vessels. Typically, these systems require the addition of serum to media, however, serum-free microcarrier systems have been reported (U.S. Pat. No. 7,524,676).

A previous disclosure describes a dengue virus manufacturing process with benzonase addition to a bioreactor prior to harvest (WO2018/183429). Further, a previous disclosure describes an open-system, dengue viral production process (WO2017/041156).

### SUMMARY OF THE INVENTION

Herein, we describe and disclose a closed-system, upstream dengue virus production process.

The present invention provides a closed-system manufacturing process for the production of dengue virus comprising: a) adherent cell expansion in one or more closed-system containers; b) a final cell expansion in a closed-system bioreactor; c) virus infection and virus production in the closed-system bioreactor; and d) virus harvest.

In an embodiment, the adherent cell expansion comprises one or more cell passages.

In another embodiment, the adherent cell expansion in one or more closed-system containers comprises one or more medium exchange steps.

In another embodiment, the adherent cell expansion comprises at least 10 cell passages, or at least 8 cell passages, or at least 6 cell passages, or at least 4 cell passages, or at least 2 cell passages.

In another embodiment, the adherent cells are grown in serum-free medium.

In another embodiment, the adherent cells are Vero cells.

In another embodiment, the one or more closed-system containers are selected from static cell culture containers.

In another embodiment, the static cell culture containers are selected from closed-system CellSTACKs® (Corning: Corning, New York) containers, closed-system cell factory systems containers, and closed-system HYPERStacks® (Corning: Corning, New York) containers.

In another embodiment, the one or more closed-system containers are closed-system CellSTACKs® containers.

In another embodiment, the adherent cell expansion occurs over 2-20 days, or 3-15 days, or 2-10 days, or 2-5 days, or 2-4 days, or 2-3 days.

In another embodiment, the adherent cell expansion occurs at a temperature of 37±2° C. and at 5%±2% CO₂.

In another embodiment, the adherent cell expansion occurs at a temperature of 37±1° C. and at 5%±1% CO₂.

In another embodiment, the closed-system bioreactor contains microcarriers and medium to support the growth of the adherent cells in the bioreactor.

In another embodiment, the mircocarriers are dextran microcarriers. In another embodiment the dextran microcarriers are Cytodex® 1 (Sigma-Aldrich: St. Louis, MO) Gamma microcarriers.

In another embodiment, the medium is serum-free medium.

In another embodiment, the medium is supplemented with Polaxamer 188.

In another embodiment, the final cell expansion in the closed-system bioreactor occurs over 120±12 hours.

In another embodiment, the final cell expansion in the closed-system bioreactor occurs at a pH range of 7.1 to 7.5 or at a pH range of 7.05 to 7.55.

In another embodiment, the final cell expansion in the closed-system bioreactor occurs at a pH of 7.3±0.05.

In another embodiment, the final cell expansion in the closed-system bioreactor occurs at a temperature of 37±2° C.

In another embodiment, the final cell expansion in the closed-system bioreactor occurs at a temperature of 37±1° C.

In another embodiment, the virus infection comprises adding virus to the closed-system bioreactor in a closed-system environment.

In another embodiment, virus production occurs at a pH range of 6.8 to 7.3 or at a pH range of 6.75 to 7.35.

In another embodiment, virus production occurs at a pH of 7.0±0.05.

In another embodiment, virus production occurs at a temperature of 34±2° C.

In another embodiment, virus production occurs at a temperature of 34±1° C.

In another embodiment, the virus harvest occurs at a temperature of 5±4° C.

In another embodiment, the virus harvest occurs at a temperature of 5±3° C.

In another embodiment, the virus harvest occurs in a closed-system environment.

In another embodiment, the virus is selected from: a primary dengue viral isolate directly obtained from an infected individual, a genetically engineered attenuated dengue virus, a genetically-engineered replication-deficient dengue virus, a cell line passaged adapted dengue virus, a cold-adapted dengue virus, a temperature-sensitive mutant dengue virus, and a genetically engineered re-assortant dengue virus.

In another embodiment, the virus is selected from DENV1, DENV2, DENV3 and DENV4.

In another embodiment, the virus is selected from rDENV1Δ30, rDENV2/4Δ30, rDENV3Δ30/Δ31 and rDENV4Δ30.

In an embodiment, the present invention provides a dengue virus vaccine manufactured by the process described herein.

In another embodiment the dengue virus vaccine is quadrivalent and consists of the four genetically attenuated viral strains rDENV1Δ30, rDENV2/4Δ30, rDENV3Δ30/31, rDENV4Δ30.

### BRIEF DESCRIPTION OF THE DRAWINGS

FIG. 1 shows a schematic representation of the closed-system, upstream dengue virus manufacturing process.

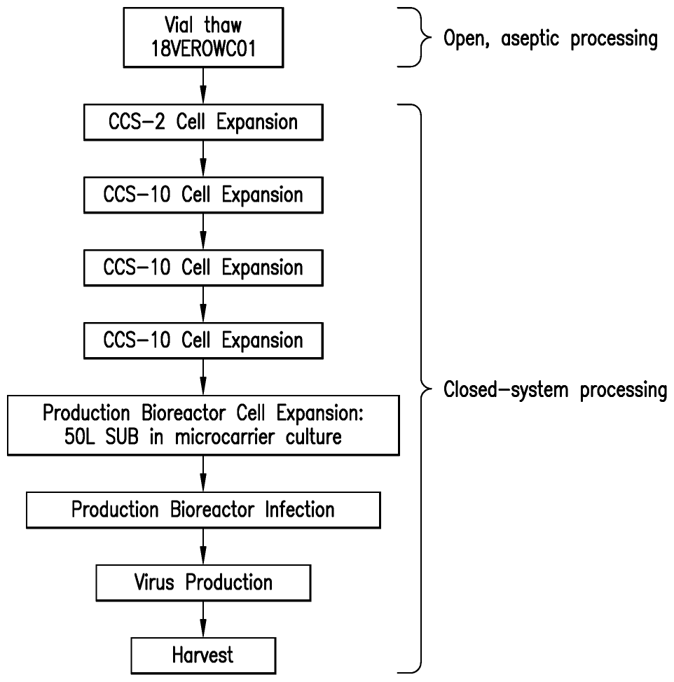

### DETAILED DESCRIPTION OF THE INVENTION

In a specific embodiment, the present invention provides a closed-system, upstream manufacturing process for the production of dengue virus comprising:
a) Vero cell incubation and growth in one or more closed-system containers for about 96±12 hours at 37±1° C. at 5%±1% CO₂;
b) medium exchange and continued incubation and growth of Vero cells in the one or more closed-system containers for about 72±12 hours at 37±1° C. at 5%±1% CO₂;
c) Vero cell harvest and plant of the Vero cells into one or more closed-system containers for about 96±12 hours at 37±1° C. at 5%±1% CO₂ for continued incubation and growth of Vero cells;
d) medium exchange and continued incubation and growth of Vero cells in the one or more closed-system containers for about 72±12 hours at 37±1° C. at 5%±1% CO₂;
e) Vero cell harvest and plant of the Vero cells into one or more closed-system containers for about 96±12 hours at 37±1° C. at 5%±1% CO₂ for continued incubation and growth of Vero cells;
f) medium exchange and continued incubation and growth of Vero cells in the one or more closed-system containers for about 48±12 hours at 37±1° C. at 5%±1% CO₂;
g) Vero cell harvest and plant of the Vero cells into one or more closed-system containers for about 96±12 hours at 37±1° C. at 5%±1% CO₂ for continued incubation and growth of Vero cells;
h) medium exchange and continued incubation and growth of Vero cells in the one or more closed-system containers for about 48±12 hours at 37±1° C. at 5%±1% CO₂;
i) Vero cell harvest and plant of the Vero cells into one or more closed-system bioreactors, each closed-system bioreactor containing microcarriers and medium, for about 120±12 hours for continued incubation and growth of Vero cells;
j) medium exchange in preparation for dengue virus infection of Vero cells;
k) dengue virus addition to the one or more bioreactors and infection of Vero cells;
l) dengue virus production; and
m) dengue virus harvest.

In an embodiment, the Vero cell harvest occurs via trypsinization.

In a specific embodiment, the present invention provides a closed-system, upstream manufacturing process for the production of dengue virus comprising:
a) Vero cell incubation and growth in one or more closed-system CCS2 CellSTACKs® for about 96±12 hours at 37±1° C. at 5%±1% CO₂;
b) medium exchange and continued incubation and growth of Vero cells in the one or more closed-system CCS2 CellSTACKs® for about 72±12 hours at 37±1° C. at 5%±1% CO₂;
c) Vero cell harvest and plant of the Vero cells into one or more closed-system CCS10 CellSTACKs® for about 96±12 hours at 37±1° C. at 5%±1% CO₂ for continued incubation and growth of Vero cells;
d) medium exchange and continued incubation and growth of Vero cells in the one or more closed-system CCS10 CellSTACKs® for about 72±12 hours at 37±1° C. at 5%±1% CO₂;
e) Vero cell harvest and plant of the Vero cells into one or more closed-system CCS10 CellSTACKs® for about 96±12 hours at 37±1° C. at 5%±1% CO₂ for continued incubation and growth of Vero cells;
f) medium exchange and continued incubation and growth of Vero cells in the one or more closed-system CCS10 CellSTACKs® for about 48±12 hours at 37±1° C. at 5%±1% CO₂;
g) Vero cell harvest and plant of the Vero cells into one or more closed-system CCS10 CellSTACKs® for about 96±12 hours at 37±1° C. at 5%±1% CO₂ for continued incubation and growth of Vero cells;
h) medium exchange and continued incubation and growth of Vero cells in the one or more closed-system CCS10 CellSTACKs® for about 48±12 hours at 37±1° C. at 5%±1% CO₂;
i) Vero cell harvest and plant of the Vero cells into one or more closed-system bioreactors, each closed-system bioreactor containing microcarriers and medium, for about 120±12 hours for continued incubation and growth of Vero cells;
j) medium exchange in preparation for dengue virus infection of Vero cells;
k) dengue virus addition to the one or more bioreactors and infection of Vero cells;
l) dengue virus production; and
m) dengue virus harvest.

In an embodiment, the Vero cell harvest occurs via trypsinization.

In a specific embodiment, the present invention provides a closed-system, upstream manufacturing process for the production of dengue virus comprising:
a) Vero cell incubation and growth in one closed-system CCS2 CellSTACK® for about 96±12 hours at 37±1° C. at 5%±1% CO₂;
b) medium exchange and continued incubation and growth of Vero cells in the closed-system CCS2 CellSTACK® for about 72±12 hours at 37±1° C. at 5%±1% CO₂;
c) Vero cell harvest and plant of the Vero cells into one or two closed-system CCS10 CellSTACKs® for about 96±12 hours at 37±1° C. at 5%±1% CO₂ for continued incubation and growth of Vero cells;
d) medium exchange and continued incubation and growth of Vero cells in the one or two closed-system CCS10 CellSTACKs® for about 72±12 hours at 37±1° C. at 5%±1% CO₂;
e) Vero cell harvest and plant of the Vero cells into two closed-system CCS10 CellSTACKs® for about 96±12 hours at 37±1° C. at 5%±1% CO₂ for continued incubation and growth of Vero cells;
f) medium exchange and continued incubation and growth of Vero cells in the two closed-system CCS10 CellSTACKs® for about 48±12 hours at 37±1° C. at 5%±1% CO₂;
g) Vero cell harvest and plant of the Vero cells into 4-6 closed-system CCS10 CellSTACKs® for about 96±12 hours at 37±1° C. at 5%±1% CO₂ for continued incubation and growth of Vero cells;
h) medium exchange and continued incubation and growth of Vero cells in the 4-6 closed-system CCS10 CellSTACKs® for about 48±12 hours at 37±1° C. at 5%±1% CO₂;
i) Vero cell harvest and plant of the Vero cells into a closed-system bioreactor, the closed-system bioreactor containing microcarriers and medium, for about 120±12 hours for continued incubation and growth of Vero cells;
j) medium exchange in preparation for dengue virus infection of Vero cells;
k) dengue virus addition to the closed-system bioreactor and infection of Vero cells;
l) dengue virus production; and
m) dengue virus harvest.

In an embodiment, the Vero cell harvest occurs via trypsinization.

In a specific embodiment, the present invention provides a closed-system, upstream manufacturing process for the production of dengue virus comprising:
a) Vero cell incubation and growth in one or more closed-system containers for about 120±12 hours at 37±1° C. at 5%±1% CO₂;
b) medium exchange and continued incubation and growth of Vero cells in the one or more closed-system containers for about 48±12 hours at 37±1° C. at 5%±1% CO₂;
c) Vero cell harvest and plant of the Vero cells into one or more closed-system containers for about 120±12 hours at 37±1° C. at 5%±1% CO₂ for continued incubation and growth of Vero cells;
d) medium exchange and continued incubation and growth of Vero cells in the one or more closed-system containers for about 48±12 hours at 37±1° C. at 5%±1% CO₂;
e) Vero cell harvest and plant of the Vero cells into one or more closed-system containers for about 120±12 hours at 37±1° C. at 5%±1% CO₂ for continued incubation and growth of Vero cells;
f) medium exchange and continued incubation and growth of Vero cells in the one or more closed-system containers for about 24±12 hours at 37±1° C. at 5%±1% CO₂;
g) Vero cell harvest and plant of the Vero cells into one or more closed-system containers for about 120±12 hours at 37±1° C. at 5%±1% CO₂ for continued incubation and growth of Vero cells;
h) medium exchange and continued incubation and growth of Vero cells in the one or more closed-system containers for about 24±12 hours at 37±1° C. at 5%±1% CO₂;
i) Vero cell harvest and plant of the Vero cells into one or more closed-system bioreactors, each closed-system bioreactor containing microcarriers and medium, for about 120±12 hours for continued incubation and growth of Vero cells;
j) medium exchange in preparation for dengue virus infection of Vero cells;
k) dengue virus addition to the one or more bioreactors and infection of Vero cells;
l) dengue virus production; and
m) dengue virus harvest.

In an embodiment, the Vero cell harvest occurs via trypsinization.

In a specific embodiment, the present invention provides a closed-system, upstream manufacturing process for the production of dengue virus comprising:
a) Vero cell incubation and growth in one or more closed-system CCS2 CellSTACKs® for about 120±12 hours at 37±1° C. at 5%±1% CO CO₂;
b) medium exchange and continued incubation and growth of Vero cells in the one or more closed-system CCS2 CellSTACKs® for about 48±12 hours at 37±1° C. at 5%±1% CO₂;
c) Vero cell harvest and plant of the Vero cells into one or more closed-system CCS10 CellSTACKs® for about 120±12 hours at 37±1° C. at 5%±1% CO₂ for continued incubation and growth of Vero cells;
d) medium exchange and continued incubation and growth of Vero cells in the one or more closed-system CCS10 CellSTACKs® for about 48±12 hours at 37±1° C. at 5%±1% CO₂;
e) Vero cell harvest and plant of the Vero cells into one or more closed-system CCS10 CellSTACKs® for about 120±12 hours at 37±1° C. at 5%±1% CO₂ for continued incubation and growth of Vero cells;
f) medium exchange and continued incubation and growth of Vero cells in the one or more closed-system CCS10 CellSTACKs® for about 24±12 hours at 37±1° C. at 5%±1% CO₂;
g) Vero cell harvest and plant of the Vero cells into one or more closed-system CCS10 CellSTACKs® for about 120±12 hours at 37±1° C. at 5%±1% CO₂ for continued incubation and growth of Vero cells;
h) medium exchange and continued incubation and growth of Vero cells in the one or more closed-system CCS10 CellSTACKs® for about 24±12 hours at 37±1° C. at 5%±1% CO₂;
i) Vero cell harvest and plant of the Vero cells into one or more closed-system bioreactors, each closed-system bioreactor containing microcarriers and medium, for about 120±12 hours for continued incubation and growth of Vero cells;
j) media exchange in preparation for dengue virus infection of Vero cells;
k) dengue virus addition to the one or more bioreactors and infection of Vero cells;
l) dengue virus production; and
m) dengue virus harvest.

In an embodiment, the Vero cell harvest occurs via trypsinization.

In a specific embodiment, the present invention provides a closed-system, upstream manufacturing process for the production of dengue virus comprising:
a) Vero cell incubation and growth in one closed-system CCS2 CellSTACK® for about 120±12 hours at 37±1° C. at 5%±1% CO₂;
b) medium exchange and continued incubation and growth of Vero cells in the closed-system CCS2 CellSTACK® for about 48±12 hours at 37±1° C. at 5%±1% CO₂;
c) Vero cell harvest and plant of the Vero cells into one or two closed-system CCS10 CellSTACKs® for about 120±12 hours at 37±1° C. at 5%±1% CO₂ for continued incubation and growth of Vero cells;
d) medium exchange and continued incubation and growth of Vero cells in the one or two closed-system CCS10 CellSTACKs® for about 48±12 hours at 37±1° C. at 5%±1% CO₂;
e) Vero cell harvest and plant of the Vero cells into two closed-system CCS10 CellSTACKs® for about 120±12 hours at 37±1° C. at 5%±1% CO₂ for continued incubation and growth of Vero cells;
f) medium exchange and continued incubation and growth of Vero cells in the two closed-system CCS10 CellSTACKs® for about 24±12 hours at 37±1° C. at 5%±1% CO₂;
g) Vero cell harvest and plant of the Vero cells into 4-6 closed-system CCS10 CellSTACKs® for about 120±12 hours at 37±1° C. at 5%±1% CO₂ for continued incubation and growth of Vero cells;
h) medium exchange and continued incubation and growth of Vero cells in the 4-6 closed-system CCS10 CellSTACKs® for about 24±12 hours at 37±1° C. at 5%±1% CO₂;
i) Vero cell harvest and plant of the Vero cells into a closed-system bioreactor, the closed-system bioreactor containing microcarriers and medium, for about 120±12 hours for continued incubation and growth of Vero cells;
j) medium exchange in preparation for dengue virus infection of Vero cells;
k) dengue virus addition to the closed-system bioreactor and infection of Vero cells;
l) dengue virus production; and
m) dengue virus harvest.

In an embodiment, the Vero cell harvest occurs via trypsinization.

In an aspect of the various embodiments, Vero cells are transferred from closed-system containers to closed-system containers in a closed-system environment.

In an aspect of the various embodiments, Vero cells are transferred from closed-system containers to closed-system bioreactors in a closed-system environment.

In an aspect of the various embodiments, dengue virus addition occurs in a closed-system environment.

In an aspect of the various embodiments, step i) occurs at a pH between 7.0-7.4 and at a temperature between 36.0-38° C. In a further embodiment, the pH is 7.3 and the temperature is at 37° C.

In another embodiment, the dengue virus is a dengue virus from one of the 4 dengue virus serotypes, referred herein as DENV1 (dengue virus serotype 1), DENV2 (dengue virus serotype 2), DENV3 (dengue virus serotype 3), or DENV4 (dengue virus serotype 4).

In one embodiment, the dengue virus comprises a viral genome that comprises a TL-2 Δ30 modification in the 3′ untranslated region (UTR). In another embodiment, the dengue virus is a DEN1 virus wherein the viral genome of DEN1 comprises a 30 nucleotide (nt) deletion corresponding to the TL2 stem-loop structure in the 3′ UTR (rDENV1Δ30) (A Live, Attenuated Dengue Virus Type 1 Vaccine Candidate with a 30-Nucleotide Deletion in the 3′ Untranslated Region Is Highly Attenuated and Immunogenic in Monkeys. Stephen S. Whitehead, Barry Falgout, Kathryn A. Hanley, Joseph E. Blaney, Jr., Lewis Markoff, Brian R. Murphy. Journal of Virology January 2003, 77 (2) 1653-1657; DOI: 10.1128/JVI.77.2.1653-1657.2003.). See also WO2003/092592 and U.S. Pat. No. 8,337,860. In another embodiment, the dengue virus is a DEN2 virus comprising the DEN2 prM and E genes on a DEN4 backbone, wherein the DEN4 backbone comprises a 30-nt deletion corresponding to the TL2 stem-loop structure in the 3′ UTR (rDENV2/4Δ30) (Anna P. Durbin, Julie H. McArthur, Jennifer A. Marron, Joseph E. Blaney, Bhavin Thumar, Kimberli Wanionek & Brian R. Murphy (2006) rDEN2/4Δ30(ME), a Live Attenuated Chimeric Dengue Serotype 2 Vaccine, is Safe and Highly Immunogenic in Healthy Dengue-Naïve Adults, Human Vaccines, 2:6, 255-260). See also WO2003/092592 and U.S. Pat. No. 8,337,860. In a further embodiment, the dengue virus is a DEN3 virus wherein the DEN3 viral genome comprises a 30 nt deletion corresponding to the TL2 stem-loop structure in the 3′ UTR and a separate, noncontiguous, upstream 31 nucleotide deletion corresponding to the TL-3 structure of the 3′ UTR (rDENV3Δ30/Δ31) (Blaney J E Jr, Sathe N S, Goddard L, et al. Dengue virus type 3 vaccine candidates generated by introduction of deletions in the 3′ untranslated region (3′-UTR) or by exchange of the DENV-3 3′-UTR with that of DENV-4. Vaccine. 2008; 26(6):817-828. doi:10.1016/j.vaccine.2007.11.082). See also WO2003/092592 and U.S. Pat. No. 8,337,860. In an additional embodiment, the dengue virus is a DEN4 virus, wherein the DEN4 viral genome comprises a 30 nucleotide deletion corresponding to the TL2 stem-loop structure in the 3′ UTR (rDEN4VΔ30) (Dengue type 4 virus mutants containing deletions in the 3′ noncoding region of the RNA genome: analysis of growth restriction in cell culture and altered viremia pattern and immunogenicity in rhesus monkeys. R Men, M Bray, D Clark, R M Chanock, C J Lai. Journal of Virology June 1996, 70 (6) 3930-3937). See also WO2003/092592 and U.S. Pat. No. 8,337,860.

The designation “rDENV1Δ30-1545” refers to a recombinant dengue 1 virus wherein the viral genome comprises (1) a 30 nt deletion of the TL2 stem-loop structure of the 3′ UTR and (2) a substitution at nucleotide position 1545 to G, which occurred after adaptation of the virus to growth in Vero cells. This virus is described in International Patent Publication WO2019/112921.

The designation “rDENV2/4Δ30(ME)-1495,7163” refers to a recombinant chimeric dengue 2/4 virus, wherein the viral genome comprises: (1) a dengue 4 backbone (C, NS1, NS2A, NS2B, NS3, NS4A, NS4B, NS5 genes) comprising (i) a 30 nt deletion of the TL2 stem-loop structure of the 3′ UTR, and (ii) substitutions at nucleotide position 1495 to U and 7163 to C, which occurred after adaptation of the virus to growth in Vero cells, and (2) dengue 2 prM and E genes. This virus is described in International Patent Publication WO2019/112921.

The designation “rDENV3Δ30/31-7164” refers to a recombinant dengue 3 virus wherein the viral genome comprises: (1) a 30 nt deletion of the TL2 stem-loop structure of the 3′ UTR, (2) a separate, 31 nt deletion in the 3′UTR, upstream of the Δ30 mutation, that deletes the TL-3 structure and (3) a substitution at nucleotide position 7164 to C, which occurred after adaptation of the virus to growth in Vero cells. This virus is described in International Patent Publication WO2019/112921.

The designation “rDENV4Δ30-7132,7163,8308” refers to a recombinant dengue 4 virus wherein the viral genome comprises: (1) a 30 nt deletion of the TL2 stem-loop structure of the 3′ UTR and (2) substitutions at nucleotide position 7132 to U, 7163 to C and 8308 to G, which occurred after adaptation of the virus to growth in Vero cells. This virus is described in International Patent Publication WO2019/112921.

In another embodiment, the dengue virus is selected from the following dengue viruses in the Dengvaxia® vaccine and the TAK-003 vaccine (formerly known as DENVax).

Dengvaxia® was released by Sanofi Pasteur in 2014. This vaccine consists of a chimeric Yellow Fever Virus with dengue premembrane and envelope structural proteins. (Thomas S J, Yoon I K. A review of Dengvaxia®: development to deployment. Hum Vaccin Immunother. 2019; 15(10):2295-2314. doi:10.1080/21645515.2019.1658503; Ee Leen Pang, Hwei-San Loh. Towards development of a universal dengue vaccine—How close are we? Asian Pacific Journal of Tropical Medicine. Volume 10, Issue 3, 2017, Pages 220-228, ISSN 1995-7645, https://doi.org/10.1016/j.apjtm.2017.03.003). Dengvaxia®, however, has had variable efficacy and safety responses especially in seronegative individuals leading to a recommendation by the WHO in 2016 to limit the introduction of the vaccine to areas with high seroprevalence. (Thomas S J, Yoon I K. A review of Dengvaxia®: development to deployment. Hum Vaccin Immunother. 2019; 15(10):2295-2314. doi:10.1080/21645515.2019.1658503).

Takeda has reported their dengue vaccine candidate TAK-003, formerly known as DENVax (Ee Leen Pang, Hwei-San Loh. Towards development of a universal dengue vaccine—How close are we? Asian Pacific Journal of Tropical Medicine. Volume 10, Issue 3, 2017, Pages 220-228, ISSN 1995-7645, doi.org/10.1016/j.apjtm.2017.03.003). This tetravalent dengue vaccine uses an attenuated dengue 2 strain. Serotypes 1, 3 and 4 are represented by replacing the envelope and premembrane genes of the TDV-2 dengue 2 strain with genes from wild-type DENV 1, DENY 3, and DENY 4 strains. (Biswal et al. Efficacy of a tetravalent dengue vaccine in healthy children aged 4-16 years: a randomised, placebo-controlled, phase 3 trial. The Lancet. Volume 395, Issue 10234, P1423-1433, May, 2020).

The dengue virus may be a primary viral isolate directly obtained from an infected individual, a genetically engineered attenuated virus, a genetically-engineered replication-deficient virus, a cell line passaged adapted virus, a cold-adapted virus, a temperature-sensitive mutant virus, or a genetically engineered re-assortant virus.

As used throughout the specification and in the appended claims, the singular forms “a,” “an,” and “the” include the plural reference unless the context clearly dictates otherwise.

Reference to “or” indicates either or both possibilities unless the context clearly dictates one of the indicated possibilities. In some cases, “and/or” was employed to highlight either or both possibilities.

The term “about”, when modifying the quantity (e.g., mM, or M) of a substance or composition, the percentage (v/v or w/v) of a formulation component, the pH of a solution/formulation, or the value of a parameter characterizing a step in a method, or the like refers to variation in the numerical quantity that can occur, for example, through typical measuring, handling and sampling procedures involved in the preparation, characterization and/or use of the substance or composition; through instrumental error in these procedures; through differences in the manufacture, source, or purity of the ingredients employed to make or use the compositions or carry out the procedures; and the like. In certain embodiments, “about” can mean a variation of ±0.1%, 0.5%, 1%, 2%, 3%, 4%, 5%, or 10%.

“Adherent cells” means cells that must be attached to a surface to grow (including for example containers, closed-system containers and microcarriers). Examples of adherent cells include: African green monkey kidney cell(s) (Vero), A549, and HepG2. Strong dengue replication has been reported in the A549 and HepG2 cell lines (Fink J, Gu F, Ling L, Tolfvenstam T, Olfat F, et al. (2007) Host Gene Expression Profiling of Dengue Virus Infection in Cell Lines and Patients. PLOS Neglected Tropical Diseases 1(2): e86).

“Bioreactor” means a multi- or single-use bioreactor. In particular, “bioreactor” means a single-use bioreactor. The bioreactor may be a 50 L volume single-use bioreactor, or a 250 L volume single-use bioreactor, or any volume in between 50 L and 250 L. In another embodiment, a “bioreactor” refers to Thermo Scientific Single-Use Bioreactor (S.U.B.). A “closed-system bioreactor” means a bioreactor that has been modified to allow sterile transfer or manipulation of cells or the processing of cells, for example, allowing the processing of cell culture materials such as cells and medium through sterile weldable tubing.

“Cell expansion”, or “CE”, or “amplification” means a series of consecutive cell growth and passage steps undertaken to generate the required number of cells for the final infection and viral production steps. In the instant invention, the cell expansion step occurs in a closed-system environment.

“CellSTACK®” refers to a static cell culture container. In an embodiment, “CellSTACK®” refers to Corning® CellSTACK® Culture Chambers comprising 2 (CCS-2; or a 2-layer static cell culture container), 10 (CCS-10; or a 10-layer static cell culture container) or 40 (CCS-40; or a 40-layer static cell culture container) layers for cell growth as specified in each embodiment. A “closed-system CellSTACK®” means a CellSTACK® that has been modified to allow sterile transfer or manipulation of cells or the processing of cells, for example, allowing the processing of cell culture materials such as cells and medium through sterile weldable tubing. Specifically, the “closed-system CellSTACK®” are modified to replace vent caps with double filters and weldable tubing to allow for closed-system processing. A “closed-system static cell culture container” means a static cell culture container that has been modified to allow sterile transfer or manipulation of cells or the processing of cells, for example, allowing the processing of cell culture materials such as cells and medium through sterile weldable tubing. Specifically, the “closed-system static cell culture container” are modified to replace vent caps with double filters and weldable tubing to allow for closed-system processing.

“Closed-system” means any cell culture manipulation that can be carried out without opening the cell culture vessel or container or bioreactor. Herein closed-system processing is carried out by pumping cell culture materials such as medium through sterile, weldable tubing. A “closed-system environment” means a contained (not open to the outside environment), sterile environment.

“Closed-system manufacturing process” means a process wherein cell expansion, virus infection, virus production and virus harvest is carried out in closed-system with closed-system containers.

“Container” means a cell culture vessel including CellSTACKs®, HYPERFlasks®, T-flasks and bioreactors. A “closed-system container” means a container or vessel that has been modified to allow sterile transfer or manipulation of cells or the processing of cells, for example, allow the processing of cell culture materials such as cells and medium through sterile weldable tubing.

“HYPERFlasks®” refers to a static cell culture container. In an embodiment, “HYPERFlasks®” refers to Corning® HYPERFlask® cell culture.

“Infection” refers to the addition of virus to a cell culture for the purpose of virus production. Infection occurs in a bioreactor or, in the instant invention, in the closed-system bioreactor. As an example, “infection” refers to the addition of at least one strain of the dengue virus. For example, infection can refer to the addition at least one of four vaccine or viral strains DENV1, DENV2, DENV3, and/or DENV4 or any modified variants thereof including attenuated variants. In another embodiment, “infection” refers to the addition of genetically attenuated viral strains rDENV1Δ30, rDENV2/4Δ30, rDENV3Δ30/31, and/or rDENV4Δ30. In the instant invention, the infection step occurs in a closed-system environment.

“Inoculation” or “plant” refers to the act of adding cells to a new cell culture vessel during a cell expansion process. For example adding cells to a new CellSTACK® or adding cells to the bioreactor.

“Media” or “medium” means any serum-free media. In an embodiment, “media” means OptiPro™ serum-free media.

“Media exchange” or “medium exchange” or “ME” refers to replacing some or all of the spent cell culture media in a vessel or container with new, unused cell culture media.

“Microcarrier” refers to support matrices composed of microscopic beads that bind to adherent cells and allow for the adherent cell (i.e. Vero cell) growth in bioreactors (or closed-system bioreactors of the instant invention). In an embodiment, “microcarrier(s)” refers to a dextran microcarrier(s). In an embodiment, “microcarrier(s)” refers to a Cytodex® 1 gamma irradiated microcarrier(s).

“Scalable” refers to the increase in virus production through increasing the volume of the cell culture container or vessel (i.e. bioreactor) rather than increasing the number of cell culture containers or vessels.

“Serum-free” means, without animal serum.

“Trypsinization” refers to the process of dissociating adherent cells from a cell culture vessel using a proteolytic enzyme such as trypsin or TrypLE. A proteolytic enzyme such as trypsin or TrypLE may also be referred to as a “cell-detachment agent”.

“Upstream” means the manufacturing process steps that include cell expansion, infection, virus production and harvest.

“Dengue virus” means DENV1, DENV2, DENV3, and/or DENV4 and any modified variants thereof including attenuated variants. In another embodiment, Dengue virus means the genetically attenuated viral strains rDENV1Δ30, rDENV2/4Δ30, rDENV3Δ30/31, and/or rDENV4Δ30.

“Virus harvest” or “viral harvest” refers to the act of harvesting virus-containing cell culture supernatant for the purpose of isolating virus. “Dengue virus harvest” refers to the act of harvesting dengue virus-containing cell culture supernatant for the purpose of isolating dengue virus. In the instant invention, the dengue virus harvest occurs in a closed-system environment.

“Virus production” or viral production” refers to virus amplification over a period of time from virus culture addition (i.e. infection) through virus harvest. As an example, “dengue virus production” refers to dengue virus amplification over a period of time from viral strains rDENV1Δ30, rDENV2/4Δ30, rDENV3Δ30/31, and/or rDENV4Δ30 culture addition (i.e. infection) through viral harvest. As an example, the period of time is from 1-20 days, or from 2 5 day, or from 3-10 days, or from 5-10 days. In the instant invention, the dengue virus production occurs in a closed-system bioreactor. The present invention provides a closed-system manufacturing process for the production of dengue virus comprising: a) adherent cell expansion in closed-system containers; b) a final cell expansion in a closed-system bioreactor; c) virus infection and virus production in the closed-system bioreactor; and d) virus harvest. In an embodiment, the adherent cell expansion comprises one or more cell passages. In another embodiment, the adherent cell expansion comprises at least 10 cell passages, or at least 8 cell passages, or at least 6 cell passages, or at least 4 cell passages, or at least 2 cell passages. In another embodiment, the adherent cells are grown in serum-free medium.

In another embodiment, the adherent cells are Vero cells. In another embodiment, the closed-system containers are selected from closed-system static cell culture containers.

In another embodiment, the closed-system containers are closed-system CellSTACK® containers.

In another embodiment, the adherent cell expansion occurs over 2-20 days, or 3-15 days, or 2-10 days, or 2-5 days, or 2-4 days, or 2-3 days.

In another embodiment, the adherent cell expansion occurs at a temperature of 37±1° C. and at 5%±1% CO₂. In another embodiment, the closed-system bioreactor contains microcarriers and medium to support the growth of the adherent cells in the bioreactor.

In another embodiment, the mircocarriers are Cytodex® 1 Gamma microcarriers.

In another embodiment, the medium is serum-free medium.

In another embodiment, the medium is supplemented with Polaxamer 188.

In another embodiment, the final cell expansion in the closed-system bioreactor occurs over 120±12 hours.

In another embodiment, the final cell expansion in the closed-system bioreactor occurs at a pH of 7.3±0.05.

In another embodiment, the final cell expansion in the closed-system bioreactor occurs at a temperature of 37±1° C.

In another embodiment, the virus infection comprises adding virus to the closed-system bioreactor in a closed-system environment.

In another embodiment, virus production occurs at a pH of 7.0±0.05.

In another embodiment, virus production occurs at a temperature of 34±1° C. In another embodiment, the virus harvest occurs at a temperature of 5±3° C.

In another embodiment, the virus harvest occurs in a closed-system environment.

In another embodiment, the virus is selected from: a primary dengue viral isolate directly obtained from an infected individual, a genetically engineered attenuated dengue virus, a genetically-engineered replication-deficient dengue virus, a cell line passaged adapted dengue virus, a cold-adapted dengue virus, a temperature-sensitive mutant dengue virus, and a genetically engineered re-assortant dengue virus.

In another embodiment, the virus is selected from DENV1, DENV2, DENV3 and DENV4.

In another embodiment, the virus is selected from rDENV1Δ30, rDENV2/4Δ30, rDENV3Δ30/Δ31 and rDENV4Δ30.

In an embodiment, the present invention provides a dengue virus vaccine manufactured by the process described herein.

In another embodiment the dengue virus vaccine is quadrivalent and consists of the four genetically attenuated viral strains rDENV1Δ30, rDENV2/4Δ30, rDENV3Δ30/31, rDENV4Δ30.

In a specific embodiment, the present invention provides a closed-system, upstream manufacturing process for the production of dengue virus comprising:
a) Vero cell incubation and growth in one or more closed-system containers for about 120±12 hours at 37±1° C. at 5%±1% CO₂;
b) medium exchange and continued incubation and growth of Vero cells in the one or more closed-system containers for about 48±12 hours at 37±1° C. at 5%±1% CO₂;
c) Vero cell harvest and plant of the Vero cells into one or more closed-system containers for about 120±12 hours at 37±1° C. at 5%±1% CO₂ for continued incubation and growth of Vero cells;
d) medium exchange and continued incubation and growth of Vero cells in the one or more closed-system containers for about 48±12 hours at 37±1° C. at 5%±1% CO₂;
e) Vero cell harvest and plant of the Vero cells into one or more closed-system containers for about 120±12 hours at 37±1° C. at 5%±1% CO₂ for continued incubation and growth of Vero cells;
f) medium exchange and continued incubation and growth of Vero cells in the one or more closed-system containers for about 24±12 hours at 37±1° C. at 5%±1% CO₂;
g) Vero cell harvest and plant of the Vero cells into one or more closed-system containers for about 120±12 hours at 37±1° C. at 5%±1% CO₂ for continued incubation and growth of Vero cells;
h) medium exchange and continued incubation and growth of Vero cells in the one or more closed-system containers for about 24±12 hours at 37±1° C. at 5%±1% CO₂;
i) Vero cell harvest and plant of the Vero cells into one or more closed-system bioreactors, each closed-system bioreactor containing microcarriers and medium, for about 120±12 hours for continued incubation and growth of Vero cells;
j) medium exchange in preparation for dengue virus infection of Vero cells;
k) dengue virus addition to the one or more bioreactors and infection of Vero cells;
l) dengue virus production; and
m) dengue virus harvest.

In an embodiment, the Vero cell harvest occurs via trypsinization.

In a specific embodiment, the present invention provides a closed-system, upstream manufacturing process for the production of dengue virus comprising:
a) Vero cell incubation and growth in one or more closed-system CCS2 CellSTACKs® for about 120±12 hours at 37±1° C. at 5%±1% CO₂;
b) medium exchange and continued incubation and growth of Vero cells in the one or more closed-system CCS2 CellSTACKs® for about 48±12 hours at 37±1° C. at 5%±1% CO₂;
c) Vero cell harvest and plant of the Vero cells into one or more closed-system CCS10 CellSTACKs® for about 120±12 hours at 37±1° C. at 5%±1% CO₂ for continued incubation and growth of Vero cells;
d) medium exchange and continued incubation and growth of Vero cells in the one or more closed-system CCS10 CellSTACKs® for about 48±12 hours at 37±1° C. at 5%±1% CO₂;
e) Vero cell harvest and plant of the Vero cells into one or more closed-system CCS10 CellSTACKs® for about 120±12 hours at 37±1° C. at 5%±1% CO₂ for continued incubation and growth of Vero cells;
f) medium exchange and continued incubation and growth of Vero cells in the one or more closed-system CCS10 CellSTACKs® for about 24±12 hours at 37±1° C. at 5%±1% CO₂;
g) Vero cell harvest and plant of the Vero cells into one or more closed-system CCS10 CellSTACKs® for about 120±12 hours at 37±1° C. at 5%±1% CO₂ for continued incubation and growth of Vero cells;
h) medium exchange and continued incubation and growth of Vero cells in the one or more closed-system CCS10 CellSTACKs® for about 24±12 hours at 37±1° C. at 5%±1% CO₂;
i) Vero cell harvest and plant of the Vero cells into one or more closed-system bioreactors, each closed-system bioreactor containing microcarriers and medium, for about 120±12 hours for continued incubation and growth of Vero cells;
j) medium exchange in preparation for dengue virus infection of Vero cells;
k) dengue virus addition to the one or more bioreactors and infection of Vero cells;
l) dengue virus production; and
m) dengue virus harvest.

In an embodiment, the Vero cell harvest occurs via trypsinization.

In a specific embodiment, the present invention provides a closed-system, upstream manufacturing process for the production of dengue virus comprising:
a) Vero cell incubation and growth in one closed-system CCS2 CellSTACK® for about 120±12 hours at 37±1° C. at 5%±1% CO₂;
b) medium exchange and continued incubation and growth of Vero cells in the closed-system CCS2 CellSTACKs® for about 48±12 hours at 37±1° C. at 5%±1% CO₂;
c) Vero cell harvest and plant of the Vero cells into one or two closed-system CCS10 CellSTACKs® for about 120±12 hours at 37±1° C. at 5%±1% CO₂ for continued incubation and growth of Vero cells;
d) medium exchange and continued incubation and growth of Vero cells in the one or two closed-system CCS10 CellSTACKs® for about 48±12 hours at 37±1° C. at 5%±1% CO₂;
e) Vero cell harvest and plant of the Vero cells into two closed-system CCS10 CellSTACKs® for about 120±12 hours at 37±1° C. at 5%±1% CO₂ for continued incubation and growth of Vero cells;
f) medium exchange and continued incubation and growth of Vero cells in the two closed-system CCS10 CellSTACKs® for about 24±12 hours at 37±1° C. at 5%±1% CO₂;
g) Vero cell harvest and plant of the Vero cells into 4-6 closed-system CCS10 CellSTACKs® for about 120±12 hours at 37±1° C. at 5%±1% CO₂ for continued incubation and growth of Vero cells;
h) medium exchange and continued incubation and growth of Vero cells in the 4-6 closed-system CCS10 CellSTACKs® for about 24±12 hours at 37±1° C. at 5%±1% CO₂;
i) Vero cell harvest and plant of the Vero cells into a closed-system bioreactor, the closed-system bioreactor containing microcarriers and medium, for about 120±12 hours for continued incubation and growth of Vero cells;
j) medium exchange in preparation for dengue virus infection of Vero cells;
k) dengue virus addition to the closed-system bioreactor and infection of Vero cells;
l) dengue virus production; and
m) dengue virus harvest.

In an embodiment, the Vero cell harvest occurs via trypsinization.

In an aspect of the various embodiments, Vero cells are transferred from closed-system containers to closed-system containers in a closed-system environment.

In an aspect of the various embodiments, Vero cells are transferred from closed-system containers to closed-system bioreactors in a closed-system environment.

In an aspect of the various embodiments, dengue virus addition occurs in a closed-system environment.

In an aspect of the various embodiments, step i) occurs at a pH between 7.0-7.4 and at a temperature between 36.0-38° C. In a further embodiment, the pH is 7.3 and the temperature is at 37° C.

In another embodiment, the dengue virus is a dengue virus from one of the 4 dengue virus serotypes, referred herein as DENV1 (dengue virus serotype 1), DENV2 (dengue virus serotype 2), DENV3 (dengue virus serotype 3), or DENV4 (dengue virus serotype 4). In one embodiment, the dengue virus comprises a viral genome that comprises a TL-2 Δ30 modification in the 3′UTR. In another embodiment, the dengue virus is a DEN1 virus wherein the viral genome of DEN1 comprises a 30 nt deletion corresponding to the TL2 stem-loop structure in the 3′ UTR (rDENV1Δ30) (A Live, Attenuated Dengue Virus Type 1 Vaccine Candidate with a 30-Nucleotide Deletion in the 3′ Untranslated Region Is Highly Attenuated and Immunogenic in Monkeys. Stephen S. Whitehead, Barry Falgout, Kathryn A. Hanley, Joseph E. Blaney, Jr., Lewis Markoff, Brian R. Murphy. Journal of Virology January 2003, 77 (2) 1653-1657; DOI: 10.1128/JVI.77.2.1653-1657.2003.). See also WO2003/092592 and U.S. Pat. No. 8,337,860. In another embodiment, the dengue virus is a DEN2 virus comprising the DEN2 prM and E genes on a DEN4 backbone, wherein the DEN4 backbone comprises a 30-nt deletion corresponding to the TL2 stem-loop structure in the 3′ UTR (rDENV2/4Δ30) (Anna P. Durbin, Julie H. McArthur, Jennifer A. Marron, Joseph E. Blaney, Bhavin Thumar, Kimberli Wanionek & Brian R. Murphy (2006) rDEN2/4Δ30(ME), a Live Attenuated Chimeric Dengue Serotype 2 Vaccine, is Safe and Highly Immunogenic in Healthy Dengue-Naïve Adults, Human Vaccines, 2:6, 255-260). See also WO2003/092592 and U.S. Pat. No. 8,337,860. In a further embodiment, the dengue virus is a DEN3 virus wherein the DEN3 viral genome comprises a 30 nt deletion corresponding to the TL2 stem-loop structure in the 3′ UTR and a separate, noncontiguous, upstream 31 nucleotide deletion corresponding to the TL-3 structure of the 3′ UTR (rDENV3Δ30/Δ31) (Blaney J E Jr, Sathe N S, Goddard L, et al. Dengue virus type 3 vaccine candidates generated by introduction of deletions in the 3′ untranslated region (3′-UTR) or by exchange of the DENV-3 3′-UTR with that of DENV-4. Vaccine. 2008; 26(6):817-828. doi:10.1016/j.vaccine.2007.11.082). See also WO2003/092592 and U.S. Pat. No. 8,337,860. In an additional embodiment, the dengue virus is a DEN4 virus, wherein the DEN4 viral genome comprises a 30 nucleotide deletion corresponding to the TL2 stem-loop structure in the 3′ UTR (rDEN4VΔ30) (Dengue type 4 virus mutants containing deletions in the 3′ noncoding region of the RNA genome: analysis of growth restriction in cell culture and altered viremia pattern and immunogenicity in rhesus monkeys.

R Men, M Bray, D Clark, R M Chanock, C J Lai. Journal of Virology June 1996, 70 (6) 3930-3937). See also WO2003/092592 and U.S. Pat. No. 8,337,860.

The designation “rDENV1Δ30-1545” refers to a recombinant dengue 1 virus wherein the viral genome comprises (1) a 30 nt deletion of the TL2 stem-loop structure of the 3′ UTR and (2) a substitution at nucleotide position 1545 to G, which occurred after adaptation of the virus to growth in Vero cells. This virus is described in International Patent Publication WO2019/112921.

The designation “rDENV2/4Δ30(ME)-1495,7163” refers to a recombinant chimeric dengue 2/4 virus, wherein the viral genome comprises: (1) a dengue 4 backbone (C, NS1, NS2A, NS2B, NS3, NS4A, NS4B, NS5 genes) comprising (i) a 30 nt deletion of the TL2 stem-loop structure of the 3′ UTR, and (ii) substitutions at nucleotide position 1495 to U and 7163 to C, which occurred after adaptation of the virus to growth in Vero cells, and (2) dengue 2 prM and E genes. This virus is described in International Patent Publication WO2019/112921.

The designation “rDENV3Δ30/31-7164” refers to a recombinant dengue 3 virus wherein the viral genome comprises: (1) a 30 nt deletion of the TL2 stem-loop structure of the 3′ UTR, (2) a separate, 31 nt deletion in the 3′UTR, upstream of the Δ30 mutation, that deletes the TL-3 structure and (3) a substitution at nucleotide position 7164 to C, which occurred after adaptation of the virus to growth in Vero cells. This virus is described in International Patent Publication WO2019/112921.

The designation “rDENV4Δ30-7132,7163,8308” refers to a recombinant dengue 4 virus wherein the viral genome comprises: (1) a 30 nt deletion of the TL2 stem-loop structure of the 3′ UTR and (2) substitutions at nucleotide position 7132 to U, 7163 to C and 8308 to G, which occurred after adaptation of the virus to growth in Vero cells. This virus is described in International Patent Publication WO2019/112921.

In another embodiment, the dengue virus is selected from the following dengue viruses in the Dengvaxia® vaccine and the TAK-003 vaccine (formerly known as DENVax).

Dengvaxia® was released by Sanofi Pasteur in 2014. This vaccine consisted of a chimeric Yellow Fever Virus with dengue premembrane and envelope structural proteins. (Thomas S J, Yoon I K. A review of Dengvaxia®: development to deployment. Hum Vaccin Immunother. 2019; 15(10):2295-2314. doi:10.1080/21645515.2019.1658503; Ee Leen Pang, Hwei-San Loh. Towards development of a universal dengue vaccine—How close are we? Asian Pacific Journal of Tropical Medicine. Volume 10, Issue 3, 2017, Pages 220-228, ISSN 1995-7645, doi.org/10.1016/j.apjtm.2017.03.003). Dengvaxia®, however, has had variable efficacy and safety responses especially in seronegative individuals leading to a recommendation by the WHO in 2016 to recommend the introduction of the vaccine in areas with high seroprevalence. (Thomas S J, Yoon I K. A review of Dengvaxia®: development to deployment. Hum Vaccin Immunother. 2019; 15(10):2295-2314. doi:10.1080/21645515.2019.1658503).

Takeda has reported their dengue vaccine candidate TAK-003, formerly known as DENVax (Ee Leen Pang, Hwei-San Loh. Towards development of a universal dengue vaccine—How close are we? Asian Pacific Journal of Tropical Medicine. Volume 10, Issue 3, 2017, Pages 220-228, ISSN 1995-7645, doi.org/10.1016/j.apjtm.2017.03.003). This tetravalent dengue vaccine uses an attenuated dengue 2 strain. Serotypes 1, 3 and 4 are represented by replacing the envelope and premembrane genes of the TDV-2 dengue 2 strain with genes from wild-type DENV 1, DENY 3, and DENY 4 strains. (Biswal et al. Efficacy of a tetravalent dengue vaccine in healthy children aged 4-16 years: a randomised, placebo-controlled, phase 3 trial. The Lancet. Volume 395, Issue 10234, P1423-1433, May, 2020).

The dengue virus may be a primary viral isolate directly obtained from an infected individual, a genetically engineered attenuated virus, a genetically-engineered replication-deficient virus, a cell line passaged adapted virus, a cold-adapted virus, a temperature-sensitive mutant virus, or a genetically engineered re-assortant virus.

### Closed-System Containers and Closed-System Bioreactors

Work in closed-system cell culture is well known in the art for both static culture and bioreactor systems. Static cell culture traditionally occurs in flasks which must be opened aseptically in order to perform media exchange or cell expansion processes. However, in an effort to reduce the opportunities for contamination, innovators have developed closed-system containers for static cell expansion. These containers include the system described herein where Corning CellSTACKs® have been modified to contain weldable tubing to eliminate the need to open the containers aseptically to add or replace media or perform cell expansion. In addition, HYPERStack® containers have also been used with tubing containing aseptic tubing connections to eliminate the need for open manipulations (Titus K, Klimovich V, Rothenberg M, Pardo P, Tanner A, Martin G. Closed-system cell culture protocol using HYPERStack vessels with gas permeable material technology. J Vis Exp. 2010; (45):2499. Published 2010 Nov. 29. doi:10.3791/2499). Additional rigid, static cell culture containers that have been reported to be modified to include sterile tubing for closed-system manipulation include: the Nunc Cell Factory system and the Corning CellCube (Abraham E., Ahmadian B. B., Holderness K., Levinson Y., McAfee E. (2017) Platforms for Manufacturing Allogeneic, Autologous and iPSC Cell Therapy Products: An Industry Perspective. In: Kiss B., Gottschalk U., Pohlscheidt M. (eds) New Bioprocessing Strategies: Development and Manufacturing of Recombinant Antibodies and Proteins. Advances in Biochemical Engineering/Biotechnology, vol 165. Springer, Cham. doi.org/10.1007/10_2017_14). In the field of cell therapy, cell culture bags have begun to be preferred over flasks due to the ease of adding tubing to these flexible, polymer-based bags including: Corning Cell Expansion Bags, Miltenyi Biotec MACS Cell Expansion and Cell Differentiation Bags and Saint Gobain VueLife Bags (N Fekete, A V Beland, K Campbell, S L Clark, C A Hoesli. Bags versus flasks: a comparison of cell culture systems for the production of dendritic cell-based immunotherapies. Transfusion, 58 (2018), pp. 1800-1813).

In addition to static cell culture, single-use bioreactor systems have also been adapted to allow for closed-system processing in order to reduce the chance for the introduction of contaminants (Parrish M. Galliher, Chapter 29—Single Use Technology and Equipment, Editor(s): Gunter Jagschies, Eva Lindskog, Karol Łącki, Parrish Galliher, Biopharmaceutical Processing, Elsevier, 2018, Pages 557-577, ISBN 9780081006238, doi.org/10.1016/B978-0-08-100623-8.00029-3). There are several classes of bioreactors used in cell culture including wave mixed, stirred, fixed bed and perfusion bioreactors, each of which can be modified (closed-system bioreactors) with tubing or aseptic connections to allow for closed-system processing of cell culture (Gregory T Frank, Transformation of biomanufacturing by single-use systems and technology, Current Opinion in Chemical Engineering, Volume 22, 2018, Pages 62-70, ISSN 2211-3398, doi.org/10.1016/j.coche.2018.09.006.). While there remains a lack of industry standard for aseptic connections, many single-use technologies are designed or customized by the end user leading to novel, process-specific, closed-system designs in bioprocessing (Parrish M. Galliher, Chapter 29—Single Use Technology and Equipment, Editor(s): Gunter Jagschies, Eva Lindskog, Karol Łącki, Parrish Galliher, Biopharmaceutical Processing, Elsevier, 2018, Pages 557-577, ISBN 9780081006238, doi.org/10.1016/B978-0-08-100623-8.00029-3).

### Dengue Virus Production, Vaccine Formulation and Methods of Use

Dengue virus production, vaccine formulation and methods of use are well known. A previous disclosure describes a dengue virus manufacturing process with benzonase addition to a bioreactor prior to harvest (WO2018/183429). Further, a previous disclosure describes an open-system, dengue viral production process (WO2017/041156). Herein, we describe and disclose a closed-system, upstream dengue virus vaccine production process.

### Upstream Dengue Virus Production Process Background and General Description

The instant invention discloses a closed-system, scalable, serum-free, microcarrier-based process for the manufacture of dengue virus, which can be used for vaccine production. In a particular embodiment, the dengue virus vaccine manufactured by the process of the instant invention is a quadrivalent vaccine consisting of the four genetically attenuated viral strains rDENV1Δ30, rDENV2/4Δ, rDENV3Δ30/31, rDENV4Δ30 produced by infecting adherent African green monkey kidney cells (e.g. Vero cells).

The use of an adherent cell line for virus production often necessitates the use of a serum-based process(es) in order to preserve cell viability after trypsinization, however work has been published to remove the need for animal-derived trypsin and the use of serum as a trypsin inhibitor (Schroder, M., Friedl, P. A protein-free solution as replacement for serum in trypsinization protocols for anchorage-dependent cells. Methods Cell Sci 19, 137-147 (1997)). We initially developed an open, aseptic viral production process utilizing HYPERFlasks® as a component of the upstream manufacturing process. This process was developed using serum-free media. Cell passages were performed using a well-defined trypsinization process with a soybean trypsin inhibitor used in lieu of a traditional serum-based trypsin inhibitor. The processes described in the present invention are fully serum-free processes resulting in a lower risk of contamination, better defined and more reproducible formulations, and elimination of the need for serum batch testing.

In order to reduce the risks presented by open, aseptic manipulation of HYPERFlasks®, a closed-system viral production process was developed utilizing modified containers, such as modified CellSTACKs®, as a component of the upstream manufacturing process. Modified bioreactors were utilized for the viral production phase with weldable tubing in which trypsin, trypsin inhibitor, fresh media and virus can be added without opening a growth chamber under aseptic conditions.

Additionally, a scalable process for dengue virus production was developed using a closed-system single-use bioreactor with proof-of-concept up to a 250 L scale. This bioreactor-based system has the potential to scale to larger volumes and thus eliminates the need to scale out into multiple cell process containers such as HYPERFlasks® or CellSTACKs®.

### Example 1

An open system HYPERFlask® upstream process was developed. This upstream process employs the following techniques, steps and conditions to make dengue virus. A frozen vial containing approximately 1.0 mL of the Vero Cell Bank (ATCC, CCL-81.2), was thawed in a 37° C. dry heat block (or equivalent), and used to inoculate one Corning T225 flask with vented cap. The flask was incubated in a stationary, humidified incubator at 37±1° C., 5%±0.5% CO₂ for approximately four days. Four days post vial thaw, 100% medium exchange was performed. Three days post media exchange, cells were harvested via trypsinization with TrypLE™ Select, and cell counts performed. Using the cell suspension obtained from the initial flask, new T225 flasks were inoculated at a target seeding cell density of 1.5*104 vc/cm2. These flasks were incubated in a stationary, humidified incubator at 37±1° C., 5%±0.5% CO₂ for approximately 4 days. Four days post-plant, 100% medium exchange was performed. Two days post media exchange, cells were harvested via trypsinization with TrypLE™ Select, the cell suspensions pooled, and cell counts performed. Using the pooled cell suspension, HYPERFlasks® were inoculated at a target seeding cell density of 1.0*104 vc/cm2 per HYPERFlasks®. The HYPERFlasks® were incubated in a stationary, humidified incubator at 37±1° C., 5%±0.5% CO₂ for approximately five days. Five days post plant, cells were harvested from one of the HYPERFlasks® via trypsinization with TrypLE™ Select and cell counts performed. The cell count from the harvested HYPERFlasks® was used to calculate the volume of virus seed to add to the HYPERFlasks® based on a multiplicity of infection (MOI) of 0.01 pfu/cell. The spent medium was removed from the HYPERFlasks® prior to infection. Virus-containing infection media was then added to each HYPERFlasks®. Once infected, the HYPERFlasks® were incubated in a stationary, humidified incubator at 34° C. (±1° C.), 5%±0.5% CO₂ for approximately 7-10 days.

Example 1 describes an initial process for the serum-free production of dengue virus using an open, aseptic process with a viral production phase taking place in HYPERFlasks®. The process consists of a cell expansion step taking place in T-flasks followed by trypsinization and plant into HYPERFlasks® which are then infected with a viral strain rDENV1Δ30, rDENV2/4Δ30, rDENV3Δ30/31, or rDENV4Δ30. The viral production phase for this process takes place at an optimized temperature of 34° C. This example describes a fully serum-free process for the production of dengue virus consisting of the four genetically attenuated viral strains rDENV1Δ30, rDENV2/4Δ30, rDENV3Δ30/31, rDENV4Δ30 produced by infecting adherent African green monkey kidney cells (Vero).

### Example 2

A CellSTACK® upstream process was developed. This upstream process employs the following techniques, steps and conditions to make dengue virus and served as a proof of concept for the development of the closed-system bioreactor process. Frozen vials, containing Vero Cell Bank were thawed in a 37° C. dry heat block and used to inoculate four T225 flasks at a cell density of approximately 4.4*104 vc/cm2. The T225 flasks were incubated in a stationary, humidified incubator at 37±1° C., 5%±0.5% CO₂ for approximately four days. Four days post vial thaw, 100% medium exchange was performed. The T225 flasks were incubated in a stationary, humidified incubator at 37±1° C., 5%±0.5% CO₂ for approximately three days. Seven days post plant, cells were harvested via trypsinization with TrypLE™ Select and cell counts performed using a ViCell cell counter. Using the cell suspension obtained from the initial container, two pre-gassed 2-layer Corning CellSTACKs® (CCS-2) were inoculated at a target plant cell density of 3.0*104 vc/cm2. The container was incubated in a stationary, humidified incubator at 37±1° C., 5%±0.5% CO₂ for approximately four days. Four days post plant, 100% medium exchange was performed. The CCS-2s were incubated in a stationary, humidified incubator at 37±1° C., 5%±0.5% CO₂ for approximately three days. Seven days post plant, cells were harvested via trypsinization with TrypLE™ Select. The cells were then harvested into a closed-system container and cell counts performed using a ViCell™ cell counter by removing the cells from the used CCS-2 vessel via pumping out through sterile, weldable tubing. Once the cells had been enumerated, the cell suspension obtained from the initial containers was then planted into three pre-gassed CCS-10s were inoculated at a target plant cell density of 2.0*104 vc/cm2. The CCS-10s were incubated in a stationary, humidified incubator at 37±1° C., 5%±0.5% CO₂ for approximately four days. Four days post plant, 100% medium exchange was performed. The CCS-10s were incubated in a stationary, humidified incubator at 37±1° C., 5%±0.5% CO₂ for approximately three days. Seven days post plant, cells were harvested via trypsinization with TrypLE™ Select, the cell suspensions pooled, and cell counts performed using a ViCell™ cell counter. Using the pooled cell suspension obtained from the initial containers, three pre-gassed CCS-40s were inoculated at a target plant cell density of 2.0*104 vc/cm2. The CCS-40s were incubated in a stationary, humidified incubator at 37±1° C., 5%±0.5% CO₂ for approximately four days. Four days post plant, 100% medium exchange was performed. The CCS-40s were incubated in a stationary, humidified incubator at 37±1° C., 5%±0.5% CO₂ for approximately three days (72±12 hours). Seven days post plant, cells were harvested via trypsinization with TrypLE™ Select, the cell suspensions pooled, and cell counts performed using a ViCell™ cell counter. Using the pooled cell suspension obtained from the initial vessels, four pre-gassed CCS-40s were inoculated at a target plant cell density of 2.0*104 vc/cm2. The CCS-40s were incubated in a stationary incubator at 37±1° C., 5%±0.5% CO₂ for approximately four days. Four days post plant, 100% medium exchange was performed. The CCS-40s were incubated in a stationary incubator at 37±1° C., 5%±0.5% CO₂ for approximately three days. Seven days post plant, cells were harvested from one process container (CCS-40) via trypsinization with TrypLE™ Select, the cell suspension pooled, and cell counts performed using a ViCell™ cell counter. Using the cell count obtained, the remaining containers were 100% medium exchanged and then virus-containing infection media was then added to each to infect at a target multiplicity of infection of 0.01 pfu/cell. The CCS-40s were incubated in a stationary incubator at 34±1° C., 5% CO₂ for the appropriate amount of days per serotype.

Example 2 builds upon the serum-free process described in Example 1 to attempt to reduce the number of open, aseptic processing steps required for virus production. In this example, we describe the utilization of a closed-processing (a closed-system) cell expansion step, by passaging cell material directly between CellSTACKs®, thus reducing the open manipulations required by the previous example in HYPERFlasks®. Scaling up production from HYPERflasks® to CellSTACKs® required optimization for cell plant densities, volume of TrypLE™ and PBS used during harvest to achieve optimal cells growth in CellSTACK®.

### Example 3

### Closed-System Upstream Process for Dengue Virus Production

This closed-system, upstream manufacturing process employs the following techniques, steps and conditions to make dengue virus and, ultimately a dengue virus vaccine.

### Vero Cell Amplification

Frozen vials of Vero Working Cell Bank, were thawed in a 37° C. dry heat block and used to inoculate one 2-layer Corning CellSTACK® (CCS-2) at a cell density of approximately 2.4*10⁴ vc/cm². The CCS-2 was incubated in a stationary, humidified incubator at 37±1° C., 5%±1% CO₂ for approximately four or five days. Four or five days post vial thaw, 100% medium exchange was performed. To accomplish the media exchange in the closed-system, sterile pump tubing was welded onto the CCS-2, all of the media was then removed from the vessel, and then an equal volume of new cell culture media was pumped into the vessel via sterile, weldable tubing. The CCS-2 was incubated in a stationary, humidified incubator at 37±1° C., 5%±1% CO₂ for approximately two or three days. Seven days post plant, the process vessel was harvested via trypsinization with TrypLE™ Select. The cells were then harvested into a sterile, closed-system container by removing the cells from the used CCS-2 vessel via pumping out through sterile, weldable tubing and cell counts performed using a ViCell™ cell counter. Once the cells had been enumerated, the cell suspension obtained from the initial containers was then planted into pre-gassed 10-layer Corning CellSTACK® (CCS-10) at a target plant cell density of 2.0*10⁴ vc/cm² or 1.0*10⁴ vc/cm². The vessel was incubated in a stationary, humidified incubator at 37±1° C., 5%±1% CO₂ for approximately four or five days. Four or five days post plant, 100% medium exchange was performed. To accomplish the media exchange in the closed-system, sterile pump tubing was welded onto the CCS-10, all of the media was then removed from the vessel, and then an equal volume of new cell culture media was pumped into the vessel via sterile, weldable tubing. The CCS-10 was incubated in a stationary, humidified incubator at 37±1° C., 5%±1% CO₂ for approximately three or four days. Seven days post plant, cells were harvested from the process container via trypsinization with TrypLE™ Select. The cells were then harvested into a sterile, closed-system container by removing the cells from the used CCS-10 vessel via pumping out through sterile, weldable tubing and cell counts performed using a ViCell™ cell counter. Once the cells had been enumerated, the cell suspension obtained from the initial containers was then inoculated into two pre-gassed CCS-10s at a target plant cell density of 2.0*10⁴ vc/cm² or 1.0*10⁴ vc/cm². The CCS-10 was incubated in a stationary, humidified incubator at 37±1° C., 5%±1% CO₂ for approximately four or five days. Four or five days post plant, 100% medium exchange was performed by removing all of the media from the vessel via sterile, weldable tubing, and then an equal volume of new cell culture media was pumped into the vessel via sterile, weldable tubing. The CCS-10s were incubated in a stationary, humidified incubator at 37±1° C., 5%±1% CO₂ for approximately one or two days. Six days post plant, cells from all process CCS-10s were harvested via trypsinization with TrypLE™ Select. The cells were then harvested into a sterile, closed-system container by removing the cells from the used CCS-10 vessel via pumping out through sterile, weldable tubing and cell counts performed using a ViCell™ cell counter. Once the cells had been enumerated, the cell suspension obtained from the initial containers was then inoculated into four to six pre-gassed CCS-10s at a target plant cell density of 2.0*10⁴ vc/cm² or 1.5*10⁴ vc/cm² or 1.0*10⁴ vc/cm² by pumping the cell suspension through sterile, weldable tubing. The CCS-10s were incubated in a stationary, humidified incubator at 37±1° C., 5%±1% CO₂ for approximately four or five days. Four or five days post plant, 100% medium exchange was performed by removing all of the media from the vessel via sterile, weldable tubing, and then an equal volume of new cell culture media was pumped into the vessel via sterile, weldable tubing. The CCS-10s were incubated in a stationary, humidified incubator at 37±1° C., 5%±1% CO₂ for approximately one or two days.

### Final Vero Cell Amplification

Six days post-plant of the N-1 stage, referring to the cell expansion directly preceding plant into the bioreactor, cells from all process containers were harvested via trypsinization with TrypLE™ Select (with 0.01% P188). The TrypLE™ Select (with 0.01% P188) was added to the cell culture vessel through sterile, weldable tubing and allowed to incubate at room temperature for 15 minutes. The cells were then manually disrupted from the surface to ensure removal of all remaining attached cells. The TrypLE™ Select (with 0.01% P188) was then deactivated through the addition of soybean trypsin inhibitor in cell culture media. The cells were then harvested into a sterile, closed-system container by removing the cells from the used CCS-10 vessel via pumping out through sterile, weldable tubing and cell counts performed using a ViCell™ cell counter. Once the cells had been enumerated, the cell suspension obtained from the initial containers was then inoculated into 1×50 L Thermo SUB (1.0 g/L Cytodex-1 microcarrier) at a target plant cell density of 1.3*10⁴+10% vc/cm² (or 13 viable cells per bead, cpb) by pumping the cell suspension into a subsurface addition line on the bioreactor through sterile, weldable tubing. Attachment for the bioreactor occurred on top of the batched Growth medium (supplemented OptiPro™ serum-free media) and microcarrier slurry (at a combined 92-95% of full working volume of 50 L).

### Dengue Virus Infection and Production

The bioreactor was controlled at a level of dissolved oxygen (DO) in the media of ≥50%, pH 7.30+0.05, 37.0° C. for approximately five days. Agitation was controlled at 75 RPM during attachment period and remained at 75 RPM from end of attachment until D2pp (day 2 post-plant). Agitation was increased to 85 RPM on D2pp until media exchange on D5pp. On D5pp, the production bioreactor underwent a 1×80% media exchange via settle-decant prior to infection. Media exchange was performed by ceasing agitation to the vessel, thus settling the microcarriers and pausing pH, DO, and vessel temperature controls. Sterile, weldable tubing was then attached to a port in the single-use bioreactor and 80% of the spent media was removed. Fresh supplemented OptiPro™ serum-free media of an equal volume was then pumped into the bioreactor through sterile, weldable tubing. Immediately after media exchange, the production bioreactor was controlled at DO≥50%, Temperature=34.0° C., Agitation=105 RPM, with no pH control. An appropriate amount of virus seed was thawed at 30.0° C. in a Water Bath and transferred via a closed-system process into ˜900 mL of infection medium. The working virus seed, stored frozen in 100 mL Meissner Cryovaults™ was held at 30.0° C. until fully thawed. The desired amount of virus was then pumped by weight through sterile, weldable tubing on the cryovault into a sterile bottle of ˜900 mL of infection media. This diluted virus was then added into the production bioreactor through sterile, weldable tubing via subsurface addition. Following closed-system virus addition, pH control was re-activated at a new setpoint of pH 7.00±0.05.

Significant development work occurred to move from static cell culture to a stirred-tank bioreactor viral production process. Optimization occurred for temperature of viral infection. The original infection temperature setpoint of 37° C. was optimized to an infection temperature of 34° C. In addition, optimization for the pH of viral production was taken into account, which was unable to be controlled in static culture. Maintaining a constant pH at a setpoint of 7.00±0.05 represented a statistically significant increase in process yield. In addition, mechanical considerations such as optimizing the agitation scheme for the 50 L bioreactor, the gas flow-rate and the impellor size and turndown ratio were attuned for the new closed-system bioreactor process. Additionally, the supplementation of the serum-free media was adjusted to contain additional Polaxamer 188 compared to static culture to improve cell growth and viral yield in the microcarrier-based system.

### Detailed Closed-System Upstream Process for Dengue Virus Production

### Vero Cell Amplification

### Vial Thaw (CE1)

Three 1 mL frozen vials, containing approximately 1.0 mL each of the Vero Working Cell Bank, were thawed in a 37° C. dry heat block and used to inoculate one 2-layer Corning Cell Stack (CCS-2) at a cell density of approximately 2.4*10⁴ vc/cm². The CCS-2 were incubated in a stationary, humidified incubator at 37±1° C., 5%±1% CO₂ for approximately four or five days (96±12 hours).

Working Cell Bank: A working cell bank is a stock of cells at a specific cell passage that have been expanded for the purpose of long-term storage to be used to begin the cell expansion for vaccine production.

### Medium Exchange (ME1)

Four or five days post vial thaw, 100% medium exchange was performed. To accomplish the media exchange in the closed-system, sterile pump tubing was welded onto the CCS-2, all of the media was then removed from the vessel, and then an equal volume of new cell culture media was pumped into the vessel via sterile, weldable tubing. The CCS-2 was incubated in a stationary, humidified incubator at 37±1° C., 5%±1% CO₂ for approximately two or three days

### Passage+1 (CE2)

Seven days post plant, the process vessel was harvested via trypsinization with TrypLE™ Select. The cells were then harvested into a sterile, closed-system container by removing the cells from the used CCS-2 vessel via pumping out through sterile, weldable tubing and cell counts performed using a ViCell™ cell counter. Once the cells had been enumerated, the cell suspension obtained from the initial containers was then inoculated into one to two pre-gassed 10-layer Corning Cell Stack (CCS-10) at a target plant cell density of 2.0*10⁴ vc/cm² or 1.0-1.5*10⁴ vc/cm². The vessel was incubated in a stationary, humidified incubator at 37±1° C., 5%±1% CO₂ for approximately four or five days.

### Medium Exchange (ME2)

Four or five days post plant, 100% medium exchange was performed. To accomplish the media exchange in the closed-system, sterile pump tubing was welded onto the CCS-10, all of the media was then removed from the vessel, and then an equal volume of new cell culture media was pumped into the vessel via sterile, weldable tubing. The CCS-10 was incubated in a stationary, humidified incubator at 37±1° C., 5%±1% CO₂ for approximately two or three days.

### Passage+2 (CE3)

Seven days post plant, the process vessel was harvested via trypsinization with TrypLE™ Select. The cells were then harvested into a sterile, closed-system container by removing the cells from the used CCS-10 vessel via pumping out through sterile, weldable tubing and cell counts performed using a ViCell™ cell counter. Once the cells had been enumerated, the cell suspension obtained from the initial containers was then inoculated into two pre-gassed CCS-10 at a target plant cell density of 2.0 vc/cm² or 1.0-1.5*10⁴ vc/cm². The CCS-10 was incubated in a stationary, humidified incubator at 37±1° C., 5%±1% CO₂ for approximately four or five days.

### Medium Exchange (ME3)

Four or five days post plant, 100% medium exchange was performed. To accomplish the media exchange in the closed-system, sterile pump tubing was welded onto the CCS-10, all of the media was then removed from the vessel, and then an equal volume of new cell culture media was pumped into the vessel via sterile, weldable tubing. The CCS-10 was incubated in a stationary, humidified incubator at 37±1° C., 5%±1% CO₂ for approximately one or two days.

### Passage+3 (CE4)

Six days post plant, all process CCS-10 were harvested via trypsinization with TrypLE™ Select. The cells were then harvested into a sterile, closed-system container by removing the cells from the used CCS-10 vessel via pumping out through sterile, weldable tubing and cell counts performed using a ViCell™ cell counter. Once the cells had been enumerated, the cell suspension obtained from the initial containers was then inoculated into four to six pre-gassed CCS-10s at a target plant cell density of 2.0*10⁴ vc/cm² or 1.0*10⁴ vc/cm². The CCS-10s were incubated in a stationary, humidified incubator at 37±1° C., 5%±1% CO₂ for approximately four or five days.

### Medium Exchange (ME4)

Four-five days post plant, 100% medium exchange was performed. To accomplish the media exchange in the closed-system, sterile pump tubing was welded onto the CCS-10, all of the media was then removed from the vessel, and then an equal volume of new cell culture media was pumped into the vessel via sterile, weldable tubing. The CCS-10s were incubated in a stationary, humidified incubator at 37±1° C., 5%±1% CO₂ for approximately one or two days.

### Vero Cell Final Amplification

### N-1 Harvest, Production Bioreactor (CE5) Cell Plant, and Growth Phase

Six days post-plant of the N-1 stage, referring to the cell expansion directly preceding plant into the bioreactor, all process vessels were harvested via trypsinization with TrypLE™ Select (with 0.01% P188). The TrypLE™ Select (with 0.01% P188) was added to the cell culture vessel through sterile, weldable tubing and allowed to incubate at room temperature for 15 minutes. The cells were then manually disrupted from the surface to ensure removal of all remaining attached cells. The TrypLE™ Select (with 0.01% P188) was then deactivated through the addition of soybean trypsin inhibitor in cell culture media. The cells were then harvested into a sterile, closed-system container by removing the cells from the used CCS-10 vessel via pumping out through sterile, weldable tubing and cell counts performed using a ViCell™ cell counter. Once the cells had been enumerated, the cell suspension obtained from the initial containers was then inoculated into 1×50 L Thermo SUB (1.0 g/L Cytodex-1 microcarrier) at a target plant cell density of 1.3*10⁴±10% vc/cm² (or 13 viable cells per bead, cpb) by pumping the cell suspension into a subsurface addition line on the bioreactor through sterile, weldable tubing. Attachment for the bioreactor occurred on top of the batched growth medium (supplemented OptiPro™ serum-free media containing 5 mM L-Glutamine concentration and 0.1% w/v P-188) and microcarrier slurry (at a combined 92-95% of full working volume of 50 L).

Microcarrier slurry: Gamma-irradiated Cytodex-1™ microcarrier powder hydrated to 25 g/L slurry in growth medium or phosphate-buffered saline (PBS) in order to present a target surface area for attachment and host cell growth in a stirred-tank bioreactor.

N-1: The N-1 step refers to the cell expansion step that immediately precedes cell transfer to the final container for infection and viral production.

Growth medium: growth medium refers to the fully supplemented cell growth media.

The bioreactor was controlled a level of dissolved oxygen (DO) in the media of ≥50%, pH 7.30+0.05, 37.0° C. for approximately five days (120±12 hours).

pH was controlled throughout the growth phase using carbon dioxide gas overlayed into the Bioreactor headspace and sodium carbonate base.

DO was controlled throughout the growth phase using an initial clean air gas overlayed into the bioreactor headspace, followed by displacement of air with oxygen into the headspace as cellular demand for DO increased. Additionally, sparged oxygen was provided as demand increased further.

Agitation was controlled at 75 RPM during attachment period and remained at 75 RPM from end of attachment until D2pp. Agitation was increased to 85 RPM on D2pp until media exchange on D5pp.

### Vero Cell Infection and Production

### Production Bioreactor Medium Exchange (ME5), Infection, and Virus Production

Five days post-plant, the production bioreactor underwent a 1×80% media exchange into infection medium via settle-decant prior to infection. Immediately after media exchange, the production bioreactor was controlled at DO≥50%, Temperature=34.0° C., Agitation=105 RPM, with no pH control.

Infection medium: infection medium refers to the serum-free media used in the process that has been fully supplemented (the same composition as above for the production growth media).

The virus container, containing the working virus seed, was thawed at 30.0° C. and a pre-determined amount, based on total microcarrier surface area, the current working volume of the bioreactor post media exchange, the infectivity of the particular virus seed, and a fixed multiplicity of infection (MOI) of 1300 pfu/cm2, was added by weight into ˜900 mL of infection medium, then added into the production bioreactor via subsurface addition using sterile pump tubing in a closed system. After virus addition, pH control was re-activated to a setpoint of 7.00±0.05 for each serotype.

Working Virus Seed: working virus seed refers to a stock of virus seed that has been prepared, characterized and stored for the purpose of infecting vaccine production reactors.

Infection Medium: infection medium refers to the serum-free media used in the process that has been fully supplemented (the same composition as above for the production growth media).

pH was controlled throughout the Virus production phase using sparged Carbon Dioxide gas into the Bioreactor subsurface and Sodium Carbonate base into the headspace.

DO was controlled throughout the Virus production phase using an initial Clean Air gas overlayed into the Bioreactor headspace, followed by displacement of Air with Oxygen into the headspace as cellular demand for DO increased. Additionally, sparged Oxygen was provided as demand increased further.

Agitation was controlled at 105 RPM during the entirety of the virus production period.

### Glucose Additions

Four days post-infection, the bioreactor received a one-time Glucose feed based on offline [Glucose] measurement to reach a target [Glucose] of 12 mM in the culture in order to avoid Glucose depletion in the host cells during the virus production process stage.

### Harvest

Once harvest criteria for infected culture duration had been met (5 days±12 hours post-infection for DENV1, 8 days±12 hours post-infection for DENV2 and DENV4, and 10 days±12 hours post-infection for DENV3), the culture stopped being controlled for pH and DO and was chilled to 5° C.±3° C., then harvested through a microcarrier separation bag (aka Harvestainer), and the harvested virus fluid (HVF) transferred into downstream purification.

### Example 4

### Dengue Virus Plaque Assay and Titers

The immunoplaque assay was used to determine the titer of dengue live attenuated tetravalent vaccine composed of dengue virus serotypes DENV1, DENV2, DENV3 and DENV4. Serially diluted virus solution (monovalent or tetravalent) was applied to a confluent monolayer of Vero cells, virus was left to adsorb for one hour, followed by overlay with a viscous medium to prevent convective virus spread. The infected culture was incubated for six days at 37° C. whereupon the virus replicates and spreads to adjacent cells in a repeating cycle. This creates an area of infected cells, which is termed a plaque. Plaques were visualized by incubation with a serotype-specific primary antibody and a Horse Radish Peroxidase (HRP) conjugated secondary antibody. Incubation with a colorimetric HRP substrate makes the plaques visible. Plaques were manually counted and the number of plaque forming units per milliliter (pfu/mL) of each serotype in the original sample was calculated. The overall titer of each serotype was calculated from the geometric mean of replicate experimental titers and reported as pfu/mL. For a sample's serotype titer to be considered valid, the titer of the respective serotype positive control that was assayed in parallel must fall within its acceptable range.

This assay was used to determine the final yield of the dengue virus production process. Table 1 below summarizes the peak plaque titer at time of harvest from the bioreactor as a percentage of the target titer for the upstream process. DENV1, DENV2 and DENV4 exceed the target titer, with DENV3 averaging just under the target, indicating that the serum free, closed-system process is an effective viral production process.

TABLE 1

| | | HVF Peak Plaque Titer | Day Post-Infection of |
|---|---|---|---|
| Serotype | Replicate | (% of target) | Peak HVF Titer |
| DENV1 | 1 | 580 | 5 |
| DENV1 | 2 | 520 | 6 |
| DENV1 | 3 | 490 | 5 |
| DENV1 | 4 | 490 | 5 |
| DENV1 | 5 | 310 | 5 |
| DENV1 | 6 | 300 | 4 |
| DENV2 | 1 | 200 | 8 |
| DENV2 | 2 | 140 | 8 |
| DENV2 | 3 | 120 | 8 |
| DENV2 | 4 | 98 | 8 |
| DENV3 | 1 | 150 | 7 |
| DENV3 | 2 | 110 | 9 |
| DENV3 | 3 | 100 | 9 |
| DENV3 | 4 | 92 | 8 |
| DENV3 | 5 | 90 | 9 |
| DENV3 | 6 | 75 | 9 |
| DENV3 | 7 | 55 | 9 |
| DENV4 | 1 | 440 | 8 |
| DENV4 | 2 | 410 | 9 |
| DENV4 | 3 | 290 | 9 |
| DENV4 | 4 | 220 | 7 |
| DENV4 | 5 | 200 | 7 |
| DENV4 | 6 | 180 | 7 |
| DENV4 | 7 | 140 | 8 |

1. A closed-system manufacturing process for the production of dengue virus comprising: a) initial cell expansion of adherent cells in serum-free medium in one or more closed-system containers; b) transfer of the adherent cells, in a closed-system environment, from the one or more closed-system containers to a closed-system bioreactor and final cell expansion of the adherent cells, wherein the closed-system bioreactor contains microcarriers and serum-free medium to support the growth of the adherent cells in the bioreactor; c) addition of dengue virus in a closed-system environment to the closed-system bioreactor, wherein the adherent cells are infected with dengue virus; d) production of dengue virus in the closed-system bioreactor; and e) harvest of the dengue virus.

2. The process of claim 1 wherein the initial cell expansion of adherent cells in serum-free medium comprises one or more cell passages in a closed-system environment.

3. The process of claim 2 wherein the initial cell expansion of adherent cells in serum-free medium comprises at least 4 cell passages in a closed-system environment.

4. (canceled)

5. The process according to claim 1 wherein the adherent cells are Vero cells.

6. The process according to claim 1 wherein the closed-system containers are closed-system static cell culture containers.

7. The process according to claim 1 wherein the initial cell expansion of adherent cells in serum-free medium occurs over 2-20 days.

8. The process according to claim 1 wherein the initial cell expansion of adherent cells in serum-free medium occurs at a temperature of 37±1° C. and at 5%±1% CO₂.

9. (canceled)

10. The process according to claim 1 wherein the mircocarriers are dextran microcarriers.

11. (canceled)

12. The process according to claim 1 wherein the medium contained in the closed-system bioreactor is supplemented with Poloxamer 188.

13. The process according to claim 1 wherein the final cell expansion in the closed-system bioreactor occurs over 120±12 hours.

14. The process according to claim 1 wherein the final cell expansion in the closed-system bioreactor occurs at a pH range of 7.05 to 7.55.

15. The process according to claim 1 wherein the final cell expansion in the closed-system bioreactor occurs at a temperature of 37±1° C.

16. (canceled)

17. The process according to claim 1 wherein the production of dengue virus occurs at a pH range of 6.75 to 7.35.

18. The process according to claim 1 wherein the production of dengue virus occurs at a temperature of 34±1° C.

19. The process according to claim 1 wherein the harvest of dengue virus occurs at a temperature of 5±3° C.

20. (canceled)

21. The process according to claim 1 wherein the dengue virus is selected from: a primary dengue viral isolate directly obtained from an infected individual, a genetically engineered attenuated dengue virus, a genetically engineered replication-deficient dengue virus, a cell line passaged adapted dengue virus, a cold-adapted dengue virus, a temperature-sensitive mutant dengue virus, and a genetically engineered re-assortant dengue virus.

22. The process according to claim 1 wherein the dengue virus is selected from DENV1, DENV2, DENV3 and DENV4.

23. The process according to claim 1 wherein the dengue virus is selected from rDENV1Δ30, rDENV2/4Δ30, rDENV3Δ30/Δ31 and rDENV4Δ30.

24. A dengue virus vaccine manufactured by the process of claim 1.

25. The dengue virus vaccine of claim 24 which is quadrivalent and comprises the four genetically attenuated viral strains rDENV1Δ30, rDENV2/4Δ30, rDENV3Δ30/31, and rDENV4Δ30.

26. The process according to claim 1 wherein the harvest of dengue virus occurs in a closed-system environment.

27. A closed-system, manufacturing process for the production of dengue virus comprising:
a) incubation and growth of Vero cells in one or more closed-system containers for about 120±12 hours at 37±1° C. at 5%±1% CO₂;
b) medium exchange and continued incubation and growth of the Vero cells in the one or more closed-system containers for about 48±12 hours at 37±1° C. at 5%±1% CO₂;
c) harvest and plant of the Vero cells into one or more closed-system containers for about 120±12 hours at 37±1° C. at 5%±1% CO₂ for continued incubation and growth of the Vero cells;
d) medium exchange and continued incubation and growth of the Vero cells in the one or more closed-system containers for about 48±12 hours at 37±1° C. at 5%±1% CO₂;
e) harvest and plant of the Vero cells into one or more closed-system containers for about 120±12 hours at 37±1° C. at 5%±1% CO₂ for continued incubation and growth of the Vero cells;
f) medium exchange and continued incubation and growth of Vero cells in the one or more closed-system containers for about 24±12 hours at 37±1° C. at 5%±1% CO₂;
g) harvest and plant of the Vero cells into one or more closed-system containers for about 120±12 hours at 37±1° C. at 5%±1% CO₂ for continued incubation and growth of the Vero cells;
h) medium exchange and continued incubation and growth of the Vero cells in the one or more closed-system containers for about 24±12 hours at 37±1° C. at 5%±1% CO₂;
i) harvest and plant of the Vero cells into one or more closed-system bioreactors, each closed-system bioreactor containing microcarriers and medium, for about 120±12 hours for continued incubation and growth of the Vero cells;
j) medium exchange in preparation for dengue virus infection of the Vero cells;
k) addition of dengue virus to the one or more bioreactors and infection of the Vero cells;
l) production of the dengue virus; and
m) harvest of the dengue virus.

28. The process of claim 27, wherein in steps c), e), and g) the Vero cells are harvested and planted in a closed-system environment.

29. The process of claim 27, wherein in step i) the Vero cells are harvested and planted in a closed-system environment.

30. The process of claim 27, wherein the addition of dengue virus occurs in a closed-system environment.

31. The process of claim 27, wherein the production of the dengue virus occurs in a closed-system environment.

32. The process of claim 27, wherein the harvest of the dengue virus occurs in a closed-system environment.
